(19) United States
(12) Patent Application Publication
(10) Pub. No.: US 2023/0211118 A1
(43) Pub. Date: Jul. 6, 2023

(54) Soldering Leads to Pads in Producing Basket Catheter

(71) Applicant: Biosense Webster (Israel) Ltd., Yokneam (IL)

(72) Inventors: Assaf Govari, Haifa (IL); Christopher Thomas Beeckler, Brea, CA (US); Joseph Thomas Keyes, Sierra Madre, CA (US); Athanassios Papaioannou, Los Angeles, CA (US); Kevin Justin Herrera, West Covina, CA (US)

(21) Appl. No.: 17/566,400

(22) Filed: Dec. 30, 2021

(51) Int. Cl. A61M 25/00; B23K 1/06; B23K 3/08; B23K 1/00; B23K 26/362; B23K 101/04; A61B 18/14; A61B 5/318

(52) U.S. Cl. CPC A61M 25/001; B23K 1/06; B23K 3/087; B23K 1/0008; B23K 26/362; B23K 2101/04; A61B 18/1492; A61B 5/318

(57) ABSTRACT

A system includes a fixture, a laser assembly, and a positioning assembly. The fixture is configured to hold (i) a substrate of a distal-end assembly of a catheter and (ii) a lead placed on a given solder pad disposed on the substrate, the laser assembly is configured to emit a laser beam, and the positioning assembly is configured to move the fixture, with the substrate and the lead, relative to the laser assembly, so as to mark a soldering position, at which the lead is to be attached to the given solder pad, with a laser spot of the laser beam.

### FIELD OF THE DISCLOSURE

The present disclosure relates generally to medical devices, and particularly to methods and systems for producing basket catheters.

### BACKGROUND OF THE DISCLOSURE

Various techniques for welding and/or soldering leads in expandable catheters have been published. Some of these techniques use one or more lasers in the production process.

The present disclosure will be more fully understood from the following detailed description of the examples thereof, taken together with the drawings in which:

### BRIEF DESCRIPTION OF THE DRAWINGS

FIG. 1 is a schematic, pictorial illustration of a catheter-based position-tracking and ablation system, in accordance with an example of the present disclosure;

FIG. 2A is a schematic, pictorial illustration of splines of a catheter of the system presented in FIG. 1, in accordance with an example of the present disclosure;

FIG. 2B is a schematic, pictorial illustration of a spline of the catheter, in accordance with another example of the present disclosure;

FIG. 3 is a schematic, pictorial illustrations of a system for soldering a lead to a soldering pad of a spline, in accordance with an example of the present disclosure; and

FIG. 4 is a flow chart that schematically illustrates a method for soldering a lead to a soldering pad of a spline, in accordance with an example of the present disclosure.

### DETAILED DESCRIPTION OF EXAMPLES

### Overview

Expandable catheters, such as basket catheters, typically have electrical connections disposed in their splines for exchanging signals between the proximal and distal ends of the catheter. The distal end of a basket catheter may comprise over 100 connections (e.g., over about 10 connections for each spline), each connection comprises a pair of a lead and a solder pad that are soldered (or attached using another technique) to one another. The lead may have a diameter of about 32 μm, corresponding to American Wire Gauge (AWG) 48, or any other suitable shape and size, and soldering over ten pairs of the lead and respective solder pads within the footprint of a spline is a difficult and time-consuming task, which may also result in an electrical short between two or more adjacent connections.

Examples of the present disclosure that are described hereinbelow provide techniques for soldering leads to respective solder pads using improved soldering system and method.

In some examples, a system for soldering comprises a soldering fixture, which is configured to hold (i) a substrate of a distal-end assembly (e.g., one or more splines) of a basket catheter and (ii) a lead, which is placed on a given solder pad disposed on the substrate.

In some examples, the solder pads are arranged along the spline so as to increase the distance between any adjacent pads, and therefore, to simplify the soldering process and reducing the odds of obtaining undesired electrical shorts therebetween. In the present example, the solder pads are disposed along a virtual diagonal extending along the longitudinal axis of the spline.

In some examples, the system comprises a laser assembly, which is configured to emit a laser beam, and to direct the laser beam toward the substrate of the spline.

In some examples, the system comprises a positioning assembly, which is configured to move the soldering fixture, which holds the substrate and the lead, relative to the laser assembly, so as to mark on the lead and/or the given solder pad a soldering position, at which the lead is to be attached to the given solder pad, with a laser spot of the laser beam.

In some examples, the system comprises a processor, which is configured to receive an image of the substrate of the spline having the plurality of solder pads disposed thereon. The processor is configured to apply a pattern recognition software for identifying: (i) the given solder pad from among the solder pads of the spline, and (ii) the lead positioned over the given solder pad.

In some examples, the system comprises a soldering gun, which is configured to attached (e.g., solder) the lead to the given soldering pad at the soldering position. In the present example, the soldering gun comprises an ultrasound transducer, which is configured to ultrasonically attach the lead to the given soldering pad at the soldering position by applying an ultrasound signal to the soldering position. In other examples, the soldering gun may use any other soldering technique to carry out the soldering between the lead and the given solder pad.

The disclosed techniques improve the quality of basket catheters splines, and improve the productivity and automation of such soldering processes. The disclosed techniques may be used, mutatis mutandis, for producing other sorts of catheters, and for soldering leads to respective pads in other sorts of electronic systems.

### System Description

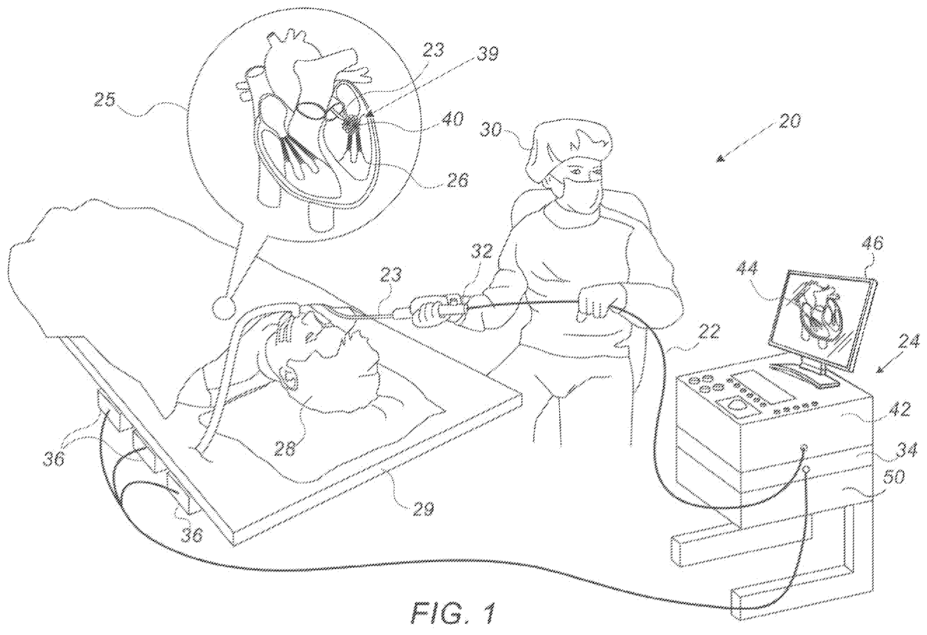

FIG. 1 is a schematic, pictorial illustration of a catheter-based position-tracking and ablation system 20, in accordance with an example of the present disclosure.

In some examples, system 20 comprises a catheter 22, in the present example an expandable cardiac catheter having a basket shape, and a control console 24. In the example described herein, catheter 22 may be used for any suitable therapeutic and/or diagnostic purposes, such as but not limited to sensing of electrocardiograms (ECGs) in tissue in question and applying ablation signals to tissue of a heart 26.

In some examples, console 24 comprises a processor 42, typically a general-purpose computer, with suitable front end and interface circuits for receiving signals from catheter 22 and for controlling other components of system 20 described herein. Processor 42 may be programmed in software to carry out the functions that are used by the system, and is configured to store data for the software in a memory 50. The software may be downloaded to console 24 in electronic form, over a network, for example, or it may be provided on non-transitory tangible media, such as optical, magnetic, or electronic memory media. Alternatively, some or all of the functions of processor 42 may be carried out using an application-specific integrated circuit (ASIC) or any suitable type of programmable digital hardware components.

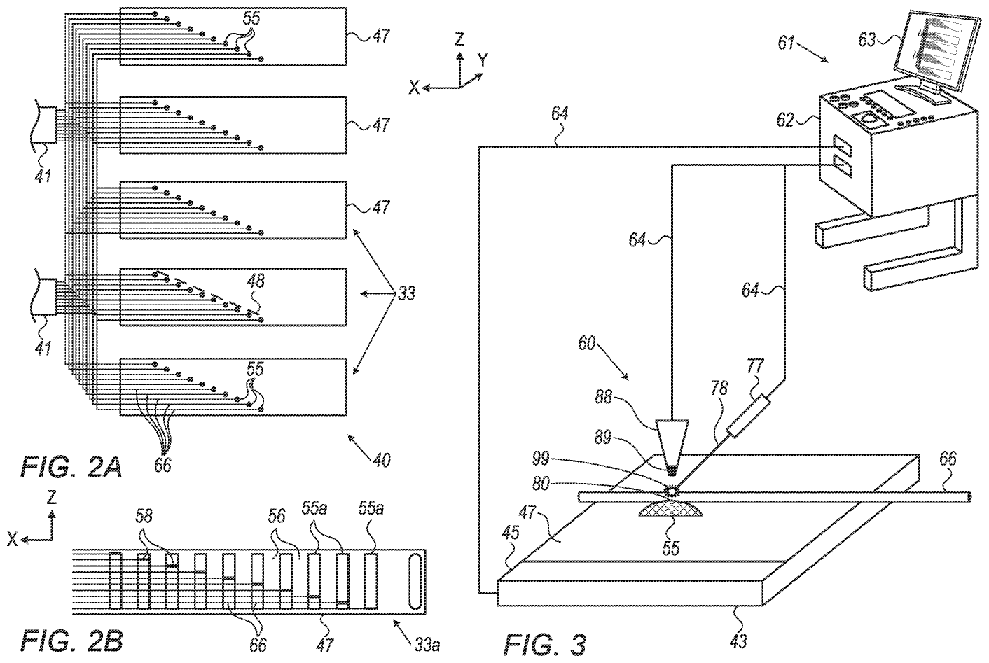

Reference is now made to an inset 25. In some examples, catheter 22 comprises a distal-end assembly 40 having multiple splines whose structure is shown in detail in FIGS. 2A and 2B below, and a shaft 23 for inserting distal-end assembly 40 to a target location for ablating tissue in heart 26. During an EA sensing and/or an ablation procedure, physician 30 inserts catheter 22 through the vasculature system of a patient 28 lying on a table 29. Physician 30 moves distal-end assembly 40 to the target location in heart 26 using a manipulator 32 near a proximal end of catheter 22, which is connected to interface circuitry of processor 42.

In some examples, catheter 22 comprises a position sensor 39 of a position tracking system, which is coupled to the distal end of catheter 22, e.g., in close proximity to distal-end assembly 40. In the present example, position sensor 39 comprises a magnetic position sensor, but in other examples, any other suitable type of position sensor (e.g., other than magnetic based) may be used.

Reference is now made back to the general view of FIG. 1. In some examples, during the navigation of distal-end assembly 40 in heart 26, processor 42 receives signals from magnetic position sensor 39 in response to magnetic fields from external field generators 36, for example, for the purpose of measuring the position of distal-end assembly 40 in heart 26. In some examples, console 24 comprises a driver circuit 34, configured to drive magnetic field generators 36. Magnetic field generators 36 are placed at known positions external to patient 28, e.g., below table 29.

In some examples, processor 42 is configured to display, e.g., on a display 46 of console 24, the tracked position of distal-end assembly 40 overlaid on an image 44 of heart 26.

The method of position sensing using external magnetic fields is implemented in various medical applications, for example, in the CARTOTM system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publications 2002/0065455 A1, 2003/0120150 A1 and 2004/0068178 A1.

In the context of the present disclosure and in the claims, the terms “about” or “approximately” for any numerical values or ranges indicate a suitable dimensional tolerance that allows the part or collection of components to function for its intended purpose as described herein.

The example configuration shown in FIG. 1 is chosen by way of example for the sake of conceptual clarity. The techniques disclosed in FIGS. 2-4 below may be applied, mutatis mutandis, using other components and settings of system 20.

### Producing Connections in Splines of a Basket Catheter

FIG. 2A is a schematic, pictorial illustration of splines 33 of distal-end assembly 40, in accordance with examples of the present disclosure. Five splines 33 (also referred to herein as spines) are presented in the example of FIG. 2A, but distal-end assembly 40 may comprise any suitable number of splines 33, e.g., about ten splines.

In some examples, splines 33 are typically similar to one another, each spline 33 comprises a substrate 47 having solder pads, also referred to herein as pads 55 disposed thereon, and leads 66 configured to be attached (e.g., soldered) to pads 55.

In some examples, each lead 66 has a circular cross section having a diameter of about 32 μm, but in other examples lead 66 may have any other suitable shape of cross-section and respective size. Lead 66 is configured to conduct signals between (i) a respective pad 55 connected thereto, and (ii) manipulator 32 and/or console 24, via one or more shielded cables 41 of leads 66 or other suitable electrically conductive wires of catheter 22, such as a long flex circuit.

In the present examples, each spline 33 comprises about ten pads 55 disposed parallel to one another along the longitudinal axis of spline 33, which is parallel to an X-axis of an XYZ coordinate system. During the production of splines 33, each lead 66 is attached (e.g., soldered) to a respective pad 55 and the high density of pads 55 and leads 66 may cause an electrical short between adjacent pairs of pads 55 and leads 66 soldered with one another.

In some examples, pads 55 are arranged along a virtual diagonal line 48, so as to increase the distance between adjacent pads 55, and thereby, to prevent or reduce the occurrence of the electrical short described above. In other examples, the increased distance between adjacent pads 55 may be obtained using any other suitable arrangement of pads 55 on the surface of substrate 47.

FIG. 2B is a schematic, pictorial illustration of a spline 33a of distal-end assembly 40, in accordance with another example of the present disclosure. In some examples, one or more splines 33a may replace one or more respective splines 33 of FIG. 2A above.

In some examples, spline 33a comprises a multi-layered structure formed on substrate 47 described in FIG. 2A above, or on any other suitable substrate. In the present example, spline 33a comprises solder pads 55a made from stripes of a flexible circuit board (flex CB) disposed parallel to the Z-axis of spline 33a.

In some examples, an electrically insulating layer 56 is disposed over substrate 47 and solder pads 55a, and leads 66 are disposed over layer 56. Moreover, spline 33a comprises slot vias 58, which are formed through layer 56, are filled with copper or with any other suitable electrically conductive substance, and are configured to conduct signals between solder pads 55a and leads 66. Note that in the example of FIG. 2B, substrate 47 appears in white color, and layer 56 is transparent for showing the structure of solder pads 55a and slot vias 58.

In the present example, slot vias 58 and solder pads 55a have approximately the same size along the X-axis of spline 33a. This configuration improves the mechanical strength of the connection between pads 55a and leads 66 of spline 33a.

### System and Method for Semi-Automatic Soldering Processes

FIG. 3 is a schematic, pictorial illustration of a system 60 for soldering leads 66 to respective soldering pads 55 disposed on substrate 47 of spline 33, in accordance with an example of the present disclosure.

In some examples, system 60 comprises a fixture, referred to herein as a soldering fixture (SF) 45 also referred to herein as a fixture for brevity, which is configured to hold: (i) substrate 47 of distal-end assembly 40, and (ii) lead 66 placed (e.g., by an operator or a robotic arm, both not shown) on solder pad 55.

In some examples, system 60 comprises a laser assembly 77, which is configured to emit a laser beam 78, in the present example toward substrate 47. Note that in the present example laser beam 78 is used for marking a position of interest, and is not used for soldering lead 66 to pad 55.

In some examples, system 60 comprises a positioning assembly (PA) 43, also referred to herein as a mount or a stage, which is configured to move SF 45 along X, Y and Z axes of the XYZ coordinate system. In the present examples, PA 43 is configured to move SF 45 with substrate 47 and lead 66, relative to laser assembly 77, so as to mark a position 80, also referred to herein as a soldering position, at which lead 66 is to be attached (e.g., soldered) to pad 55, with a laser spot 99 of laser beam 78. In other words, lead 66 is positioned over pad 55, and spot 99 marks the soldering position therebetween.

In some examples, system 60 may comprise a camera (not shown), which is configured to acquire images of the surface of substrate 47, and a processor 62 (described in detail below), which is configured to display the image acquired by the camera on a display 63, which is described below.

In some examples, processor 62 is configured to identify position 80, e.g., by applying a pattern recognition software to the image acquired by the camera.

In some examples, processor 62 is connected, via cables 64 or wirelessly to PA 43, laser assembly 77 and a soldering gun 88 (described below). Processor 62 is configured to control laser assembly 77 and PA 43 for positioning laser spot 99 on the surface of lead 66 at the identified position 80. Note that using the camera and processor 62 enables fully automatic positioning of laser spot 99 at position 80.

In such examples, PA 43 is configured to identify pad 55 among multiple pads 55 that are disposed on substrate 47 for conducting electrical signals between distal end assembly 40 and the proximal end of catheter 22. The identification of the specific pad 55 may be carried out using the pattern recognition software as described above. As an alternative, the substrate 47 can be masked with a specific pattern (e.g., a striped pattern or a green screen) so that the pads 55 can be recognized against the background pattern. Thereafter, the laser can be programmed to store the specific location of each of the pads 55 prior to applying the laser light for welding.

In other examples, at least part of the positioning of laser spot 99 may be carried out manually, e.g., without applying the pattern recognition software.

In some examples, system 60 comprises an operating console 61 having processor 62, display 63 and other components described herein. Console 61 may comprise a general-purpose computer, with suitable front end and interface circuits for receiving signals from and for controlling the components of system 60 as will be described herein. Processor 62 may be programmed in software to carry out the functions that are used by the system, and is configured to store data for the software in a memory of console 61. The software may be downloaded to console 61 in electronic form, over a network, for example, or it may be provided on non-transitory tangible media, such as optical, magnetic, or electronic memory media. Alternatively, some or all of the functions of processor 62 may be carried out using an application-specific integrated circuit (ASIC) or any suitable type of programmable digital hardware components.

In some examples, system 60 comprises soldering gun 88, which is configured to attached (e.g., solder) lead 66 to pad 55 at position 80. Soldering gun 88 is typically controlled by processor 62, and may comprise an ultrasound transducer 89, which is configured to ultrasonically bond lead 66 to pad 55 at position 80 by applying an ultrasound signal to lead 66 and pad 55 at position 80. In the context of the present disclosure and in the claims, the terms “ultrasonically bonded” and “ultrasonically attached” and their grammatical variations refer to ultrasonic techniques for welding or soldering between leads 66 and solder pads 55 of FIG. 2A above. In other examples, soldering gun 88 is configured to carry out the soldering process using any other suitable soldering and/or welding technique known in the art.

In some examples, positioning assembly 43 is configured to move at least soldering gun 88 relative to substrate 47 for aligning soldering gun 88 and laser spot 99 before applying the ultrasound signal.

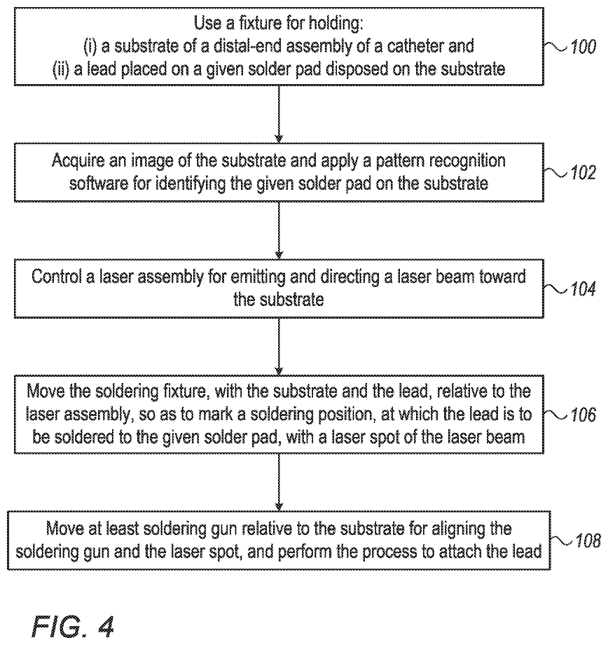

FIG. 4 is a flow chart that schematically illustrates a method for soldering lead 66 to pad 55 of spline 33, in accordance with an example of the present disclosure.

The method begins at a substrate and lead fixating step 100 with using SF 45 for holding: (i) substrate 47 of a respective spline 33 of distal-end assembly 40, and (ii) lead 66 placed on a given solder pad, e.g., pad 55 of FIG. 2A above, disposed on substrate 47, as described in detail in FIG. 3 above.

At a solder pad identification step 102, processor 62 receives an image of the surface of substrate 47 having multiple pads 55, and applied to the image a pattern recognition software configured to identify the position of (the given solder) pad 55 on substrate 47, as described in detail in FIG. 3 above.

At a laser beam directing step 104, processor 62 is configured to control laser assembly 77 for emitting and directing laser beam 78 toward substrate 47, as described in detail in FIG. 3 above.

At a fixture moving step 106, processor 62 is configured to control positioning assembly 43 to move soldering fixture 45 together with substrate 47 and lead 66, relative to laser assembly 77, so as to mark position 80, at which lead 66 is to be attached (e.g., soldered) to (the given solder) pad 55. In the present example, position 80 is marked using laser spot 99 of laser beam 78, as described in detail in FIG. 3 above.

At a soldering step 108 that concludes the method, processor 62 is configured to move at least soldering gun 88 relative to substrate 47 for aligning soldering gun 88 and laser spot 99, and subsequently, perform the process to attach lead 66 to pad 55 by applying the ultrasound signal to lead 66 and pad 55 at position 80, as described in FIG. 3 above.

The method of FIG. 4 is simplified for the sake of conceptual clarity and is provided by way of example. In other examples, the order of the steps may alter for the sake of improved productivity and/or for any suitable considerations.

### EXAMPLE 1

A system (60), including: (a) a fixture (45), which is configured to hold (i) a substrate (47) of a distal-end assembly (40) of a catheter (22) and (ii) a lead (66) placed on a given solder pad (55) disposed on the substrate (47), (b) a laser assembly (77), which is configured to emit a laser beam (78), and (c) a positioning assembly (43), which is configured to move the fixture (45), with the substrate (47) and the lead (66), relative to the laser assembly (77), so as to mark a soldering position (80), at which the lead (66) is to be attached to the given solder pad (55), with a laser spot (99) of the laser beam (78).

### EXAMPLE 2

The system according to Example 1, wherein the system includes a processor (62), which is configured to: (i) identify the given solder pad among multiple solder pads that are disposed on the substrate for conducting electrical signals between a distal end and a proximal end of the catheter, and control the positioning assembly to move the fixture for marking the soldering position.

### EXAMPLE 3

The system according to Examples 1 or 2, wherein the system includes a soldering gun (88), which is configured to attach the lead to the given soldering pad at the soldering position.

### EXAMPLE 4

The system according to Example 3, wherein the positioning assembly is configured to move at least the soldering gun relative to the substrate for aligning the soldering gun and the laser spot.

### EXAMPLE 5

The system according to Example 3, wherein the soldering gun includes an ultrasound transducer, which is configured to ultrasonically attach the lead to the given soldering pad at the soldering position by applying an ultrasound signal to the soldering position.

### EXAMPLE 6

A method, including: (a) holding: (i) a substrate (47) of a distal-end assembly (40) of a catheter (22) and (ii) a lead (66) placed on a given solder pad (55) disposed on the substrate (47), (b) emitting a laser beam (78) toward the substrate (47), and (c) moving the substrate (47) and the lead (66), relative to the laser beam (78), so as to mark a soldering position (80), at which the lead (66) is to be attached to the given solder pad (55), with a laser spot (99) of the laser beam (78).

### EXAMPLE 7

The method according to Example 6, wherein moving the substrate and the lead includes: (i) identifying the given solder pad among multiple solder pads that are disposed on the substrate for conducting electrical signals between a distal end and a proximal end of the catheter, and (ii) moving the substrate for marking the soldering position.

### EXAMPLE 8

The method according to Examples 6 or 7, and including attaching the lead to the given soldering pad at the soldering position.

### EXAMPLE 9

The method according to Example 8, wherein attaching the lead to the given soldering pad includes moving at least a soldering gun that carries out the attaching relative to the substrate for aligning the soldering gun and the laser spot.

### EXAMPLE 10

The method according to Example 8, wherein attaching the lead to the given soldering pad includes applying an ultrasound signal to the soldering position.

Although the examples described herein mainly address soldering of leads to soldering pads in basket catheters, the methods and systems described herein can also be used in other applications of soldering processes, such as in soldering leads to flex circuits which are then mounted onto balloons.

It will thus be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art.

1. A system, comprising:
a fixture, which is configured to hold (i) a substrate of a distal-end assembly of a catheter and (ii) a lead placed on a given solder pad disposed on the substrate;
a laser assembly, which is configured to emit a laser beam; and
a positioning assembly, which is configured to move the fixture, with the substrate and the lead, relative to the laser assembly, so as to mark a soldering position, at which the lead is to be attached to the given solder pad, with a laser spot of the laser beam.

2. The system according to claim 1, and comprising a processor, which is configured to: (i) identify the given solder pad among multiple solder pads that are disposed on the substrate for conducting electrical signals between a distal end and a proximal end of the catheter, and (ii) control the positioning assembly to move the fixture for marking the soldering position.

3. The system according to claim 1, and comprising a soldering gun, which is configured to attach the lead to the given soldering pad at the soldering position.

4. The system according to claim 3, wherein the positioning assembly is configured to move at least the soldering gun relative to the substrate for aligning the soldering gun and the laser spot.

5. The system according to claim 3, wherein the soldering gun comprises an ultrasound transducer, which is configured to ultrasonically attach the lead to the given soldering pad at the soldering position by applying an ultrasound signal to the soldering position.

6. A method, comprising:
holding: (i) a substrate of a distal-end assembly of a catheter and (ii) a lead placed on a given solder pad disposed on the substrate;
emitting a laser beam toward the substrate; and
moving the substrate and the lead, relative to the laser beam, so as to mark a soldering position, at which the lead is to be attached to the given solder pad, with a laser spot of the laser beam.

7. The method according to claim 6, wherein moving the substrate and the lead comprises: (i) identifying the given solder pad among multiple solder pads that are disposed on the substrate for conducting electrical signals between a distal end and a proximal end of the catheter, and (ii) moving the substrate for marking the soldering position.

8. The method according to claim 6, and comprising attaching the lead to the given soldering pad at the soldering position.

9. The method according to claim 8, wherein attaching the lead to the given soldering pad comprises moving at least a soldering gun that carries out the attaching relative to the substrate for aligning the soldering gun and the laser spot

10. The method according to claim 8, wherein attaching the lead to the given soldering pad comprises applying an ultrasound signal to the soldering position.
